(19) United States
(12) Patent Application Publication
(10) Pub. No.: US 2024/0180812 A1
(43) Pub. Date: Jun. 6, 2024

(54) METHOD OF IMPROVING OUTCOMES OF DERMAL TATTOOS AND TATTOO REMOVAL PROCEDURES

(71) Applicant: Mark E. Dillon, Center Valley, PA (US)

(72) Inventor: Mark E. Dillon, Center Valley, PA (US)

(73) Assignee: Bio Med Sciences, Inc., Allentown, PA (US)

(21) Appl. No.: 18/227,280

(22) Filed: Jul. 27, 2023

Related U.S. Application Data
(60) Provisional application No. 63/392,696, filed on Jul. 27, 2022.

(51) Int. Cl. A61K 8/891; A61K 8/02; A61Q 1/14

(52) U.S. Cl. CPC A61K 8/891; A61K 8/0233; A61Q 1/145; A61K 2800/20

(57) ABSTRACT

This invention relates to novel methods of improving the overall outcome of a dermal tattoo when it is applied, such methods including applying a silicone-based material (such as a silicone-based film or silicone ointment) to a freshly created tattoo to improve comfort, offer barrier function, promote moist healing, and provide an effect to prevent and reduce scar formation. The invention also relates to a novel method of improving the overall outcome when a dermal tattoo is removed, such method including applying a silicone-based material to the tattoo site as a pre-treatment to laser tattoo removal.

### CROSS REFERENCE TO RELATED APPLICATION

This application claims the benefit of U.S. Provisional Patent Application Ser. No. 63/392,696, which was filed on Jul. 27, 2022 and which is incorporated herein by reference in its entirety.

### FIELD OF THE INVENTION

This invention relates to the application of tattoos and the removal of tattoos, and more particularly concerns novel methods of improving the outcome of dermal tattoos when they are applied, as well as when they are removed.

### BACKGROUND OF THE INVENTION

Tattooing is an ancient practice thought to have roots with tribal identification. Essentially every culture around the world has practiced tattooing in one fashion or another for a plethora of reasons. Most common is simple aesthetic adornment, sometimes with cultural or subcultural associations. In modern times other reasons include cosmetic applications. Examples include: faux nipple areolas after mastectomy, traumatic hypopigmentation camouflage, and permanent make-up on the face. Tattoos have even been used for nefarious purposes, such as the serialized branding of whole populations.

Regardless of the reason, all tattooing involves penetrating the upper layer of the skin and depositing ink into the dermal tissue. Professional inks may be made from iron oxides, metal salts, or plastics. Homemade or traditional tattoo inks may be made from pen ink, soot, dirt, blood, or other ingredients. The effect is achieved by manually inflicting deep abrasion of the skin with a sharp instrument, or by using a vibrating mechanical needle device. Unintentional tattooing can occur traumatically by mechanisms such as blast or abrasion where debris is driven into the wound. When penetrated deep enough, the pigment particles within the ink become entrapped in the collagen structure of the dermis and therefore persist for a lifetime.

After tattooing, the skin undergoes a recovery process in two basic phases. The acute wound phase is characterized by discomfort and irritation; and may present with exudation, erythema and edema lasting a few days. The prolonged phase is the healing period during which the acute symptoms gradually subside, and the skin stabilizes over a period of weeks.

As with any human endeavor, the longevity of the decision does not always equal the permanence of the result. Not surprisingly, tattoo removal is a common procedure. The simplest, and crudest, method is dermabrasion, where the pigmented dermis is mechanically obliterated, and the site is simply allowed to heal. A more sophisticated approach is to use lasers. Laser tattoo removal works by fracturing the pigment particles in situ, so that the body can then remove the fragments over time by natural processes. Dermabrasion almost always leaves significant scarring, whereas laser removal often does not.

When the proper laser protocols and patient aftercare are utilized, it is very uncommon for a patient to scar from laser treatment.

This invention relates to acute and prolonged tattoo aftercare methods for treating newly created tattoos on a person's skin, thereby enhancing the likelihood of an optimal outcome. Additionally, the method of this invention also improves the results when a tattoo is removed by laser technique by providing a more cosmetically appealing scar-free dermal matrix for depigmentation. This can be achieved by using this inventive method to prevent scar formation at the time of tattoo application as well as a pre-treatment to laser tattoo removal to reduce existing scarring prior to laser tattoo removal.

### DESCRIPTION OF THE PRIOR ART

The known methods of acute and prolonged tattoo aftercare are as diverse as those who give and get them. They range from no-care, to ointments, to simple plastic films, and polymer films coated with adhesive.

No-care typically results in dry, slow and painful healing. Dry healing resulting in eschar formation is known to increase scar formation in wound healing. Beyond no-care, simple ointments, such as petrolatum, are applied to the tattoo application site to provide some occlusion and a moist environment. This technique has little effect on pain and can be tedious and untidy to manage. Beyond ointment, simple cellophane film, such as Saran® wrap, is sometimes applied over the tattoo application site and then taped in place. While this method is less messy and provides some occlusion, it is cumbersome and uncomfortable. Furthermore, barrier function is problematic if not secured sufficiently.

The next level of care is with polyurethane (“PU”) films that are secured in place over the tattoo application site with an acrylic pressure sensitive adhesive (“PSA”). Products used for this purpose include Dermalize Pro, Saniderm, and KeyEntre. These products provide a moist environment for wound healing and act as a barrier to contamination. With these products, barrier function is more effective and exudate leakage is largely avoided. However, PU/PSA films do little for comfort and can even be uncomfortable, particularly upon removal. Some of these products claim to reduce the likelihood of scarring. It is believed that this claim has no justification beyond simply avoiding eschar formation.

Tattoo aftercare with cellophane film or PU/PSA films typically lasts no more than two days. Also, people generally do not want to cover up a newly created tattoo for any longer than may be necessary (typically no more than a day or two) since they typically want to show off their newly created tattoos as soon as possible.

It is well known that silicone-based sheeting and gel products are effective for the prevention or reduction (together or separately “management”) of dermal scaring at wound/surgical sites. There are numerous scar management products available in the marketplace. Bio Med Sciences, Inc. of Allentown, Pennsylvania, manufactures and markets the Oleeva® and Silon® brands of scar management products including self-adhesive patches and dressings (for the purposes hereof, the term patch or dressing may be used interchangeably). My U.S. Pat. Nos. #4,832,009, #5,656,279, #5,980,923, and #7,087,135, which are incorporated herein by reference, describe wound dressings and scar management products utilizing an interpenetrating polymer network (“IPN”) of silicone and polytetrafluoroethylene.

Bio Med Sciences, Inc. also manufactures and markets other scar management and skin care products under various brands and in a variety of formats, including silicone-lined thermoplastic splinting as described in my U.S. Pat. No. 5,759,560, silicone-lined textiles as described in my U.S. Pat. No. 8,084,051, as well as silicone liquids and semisolid silicone paste applicator sticks. My U.S. Pat. No. 5,759,560 and my U.S. Pat. No. 8,084,051 also are incorporated herein by reference. Bio Med Sciences, Inc. also markets an anti-wrinkle product for the reduction in the appearance of fine rhytides as disclosed in my U.S. Pat. No. 9,414,970 B2, which is incorporated herein by reference.

Silicone-based sheeting and silicone-based gel products are not known for use in acute and prolonged tattoo aftercare of a newly created tattoo.

### SUMMARY OF THE INVENTION

In an effort to improve the art, I have discovered that the application of a silicone-based material (including silicone-based material in the form of a silicone-based film, or a silicone-based ointment, or a silicone-based liquid, or a silicone-based paste, or a silicone-based sheeting) to a freshly applied tattoo during the acute tattoo aftercare and/or the prolonged tattoo aftercare not only improves comfort, offers barrier function, and promotes moist healing, it provides an effect to cosmetically prevent and reduce scars at the tattoo site. By reducing and preventing scar formation the overall outcome of a tattoo is improved.

Further, I have discovered that, in connection with the removal of a tattoo, application of a silicone-based material (including silicone-based material in the form of a silicone-based film, or a silicone-based ointment, or a silicone-based liquid, or a silicone-based paste, or a silicone-based sheeting) to a tattoo that is to be removed as a pre-treatment to laser tattoo removal results in optimizing the aesthetic condition of the site from which the tattoo is removed after removal of the tattoo via laser tattoo removal.

### BRIEF DESCRIPTION OF THE DRAWINGS

FIG. 1 shows a schematic representation of a partial view in cross-section of a monolithic design of a preferred tattoo dressing 100 constructed in accordance with the invention, the tattoo dressing 100 comprising a silicone matrix 105.

FIG. 2 shows a schematic representation of a partial view in cross-section of a bilayer design of another preferred tattoo dressing 200 constructed in accordance with the invention, the tattoo dressing 200 having a skin contacting silicone surface 210 and an outer distal layer 220.

### DETAILED DESCRIPTION

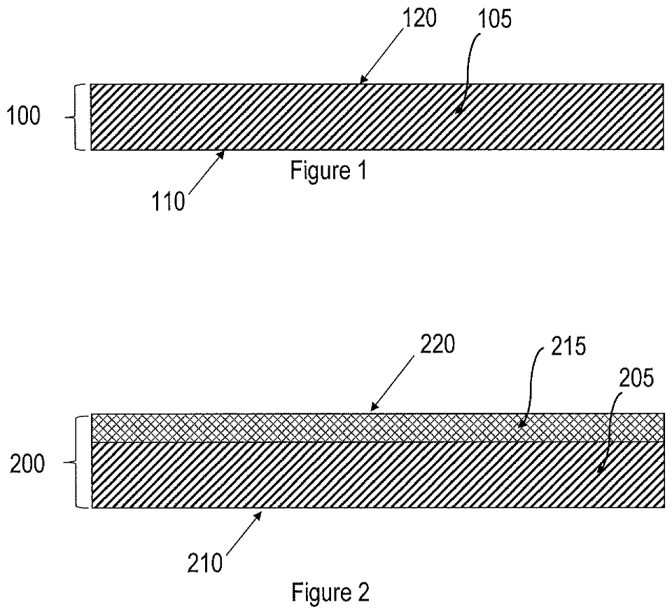

In a preferred embodiment, the invention, as illustrated in FIG. 1, comprises a dressing 100 having a silicone matrix 105. The dressing 100 has a skin contacting surface 110 and an outer surface 120. In the preferred embodiment of the invention shown in FIG. 2, the dressing 200 has a layer 205 comprised of a silicone matrix. The dressing 200 has a skin contacting surface 210, a distal non-skin contacting backing layer 215, and an outer distal surface 220.

My inventive method of acute and prolonged aftercare enhances and improves the final outcome of tattooing. For instance, it is believed that the healing environment created by my invention results in the site of a newly created tattoo healing better than the healing environment created by the aftercare currently used for tattoo aftercare, as well as resulting in better (e.g., sharper and clearer) colors in the tattoo.

Preferably, the skin contacting layer of the dressing comprises a silicone-based material, such as silicone, polydimethylsiloxane, polyorganosiloxane, silicone gel, or silicone elastomer. The dressing also may have additional layers positioned over the skin contacting layer, such as an interpenetrating polymer network (“IPN”), or a polymer that forms an interpenetrating polymer network (“IPN”) with the skin contacting layer, or an outer backing layer such as textile fabric, foam, non-woven film, mesh, thermoplastic material, or material other than textile fabric. The dressing may comprise Oleeva® scar management dressings, Silon® scar management dressings, or any of the dressings described in my U.S. Pat. Nos. 4,832,009, 5,656,279, 5,980,923, 7,087,135, 5,759,560, and 8,084,051, wherein all of such dressings have an effect of reducing and preventing scarring.

In use, the tattoo dressing of the invention may be applied to the tattoo site immediately or soon after the application of the tattoo for the acute phase of healing (that is, for acute tattoo aftercare) and, if desired, for acute tattoo aftercare and the prolonged phase of healing (that is, for prolonged tattoo aftercare), such that the silicone-based material of the skin contacting layer of the tattoo dressing is in contact with the tattoo site to provide a comfortable, occlusive barrier while preventing or reducing dermal scarring, resulting in the site of a newly created tattoo healing better than the healing environment created by the aftercare currently used for tattoo aftercare, as well as resulting in better (e.g., sharper and clearer) colors in the tattoo.

Further, in use, following the acute phase of healing (that is, following acute tattoo aftercare), the tattoo dressing of the invention may be applied to the tattoo site for the prolonged phase of healing (that is, prolonged tattoo aftercare), such that the silicone-based material of the skin contacting layer of the tattoo dressing is in contact with the tattoo site to provide a comfortable, occlusive barrier while preventing or reducing dermal scarring, resulting in the site of a newly created tattoo healing better than the healing environment created by the aftercare currently used for tattoo aftercare, as well as resulting in better (e.g., sharper and clearer) colors in the tattoo.

Also, in use, the tattoo dressing of the invention may be applied to the tattoo site as a pre-treatment to laser tattoo removal, such that the silicone-based material of the skin contacting layer of the tattoo dressing is in contact with the tattoo site to reduce existing scarring at the tattoo site prior to laser tattoo removal, resulting in optimizing the aesthetic condition of the site from which the tattoo is removed after removal of the tattoo via laser tattoo removal.

In general, in accordance with the invention, a tattoo is applied to a person's skin, and immediately or soon after the tattoo is applied, the silicone-based skin contacting surface of a silicone-based material (such a silicone-based skin contacting surface comprising the outer surface of the silicone-based material (the silicone-based material comprising, for example, a silicone-based film, or a silicone-based liquid, or a silicone-based ointment, or a silicone-based paste, or a silicone-based sheeting) that is placed over and into contact with the tattoo site) is placed over and into contact with the tattoo site, such that the silicone-based skin contacting surface of the silicone-based material is in contact with the tattoo to provide a comfortable, occlusive barrier while preventing or reducing dermal scarring. The silicone-based skin contacting surface of a silicone-based material is maintained in contact with the newly created tattoo for at least about one day and preferably for several days and more preferably for a week and even more preferably for two weeks and still even more preferably for several weeks, resulting in the site of a newly created tattoo healing better than the healing environment created by the aftercare currently used for tattoo aftercare, as well as resulting in better (e.g., sharper and clearer) colors in the tattoo, thereby enhancing and improving the final outcome of the tattoo.

Further, in general, in accordance with the invention, a tattoo is applied to a person's skin, and following the acute phase of healing (that is, following acute tattoo aftercare), the silicone-based skin contacting surface of a silicone-based material (such a silicone-based skin contacting surface comprising the outer surface of the silicone-based material (the silicone-based material comprising, for example, a silicone-based film, or a silicone-based liquid, or a silicone-based ointment, or a silicone-based paste, or a silicone-based sheeting) that is placed over and into contact with the tattoo site) is placed over and into contact with the tattoo site, such that the silicone-based skin contacting surface of the silicone-based material is in contact with the tattoo for the prolonged phase of healing (that is, prolonged tattoo aftercare) to provide a comfortable, occlusive barrier while preventing or reducing dermal scarring. The silicone-based skin contacting surface of a silicone-based material is maintained in contact with the newly created tattoo for at least about one day and preferably for several days and more preferably for a week and even more preferably for two weeks and still even more preferably for several weeks, resulting in the site of a newly created tattoo healing better than the healing environment created by the aftercare currently used for tattoo aftercare, as well as resulting in better (e.g., sharper and clearer) colors in the tattoo, thereby enhancing and improving the final outcome of the tattoo.

Also, in general, in accordance with the invention, in connection with the removal of a tattoo, the silicone-based skin contacting surface of a silicone-based material (such a silicone-based skin contacting surface comprising the outer surface of the silicone-based material (the silicone-based material comprising, for example, a silicone-based film, or a silicone-based liquid, or a silicone-based ointment, or a silicone-based paste, or a silicone-based sheeting) that is placed over and into contact with a tattoo that is to be removed) is placed over and into contact with the tattoo that is to be removed as a pre-treatment to laser tattoo removal, such that the silicone-based skin contacting surface of the silicone-based material is in contact with the tattoo that is to be removed to reduce existing scarring at the tattoo site prior to laser tattoo removal. The silicone-based skin-contacting surface of the silicone-based material is maintained in contact with the tattoo that is to be removed for at least about one day and preferably for several days and more preferably for a week and even more preferably for two weeks and still more preferably for several weeks. After this pre-treatment, the tattoo is removed using laser tattoo removal, leaving the site from where the tattoo is removed in an optimal aesthetic condition due to being tattoo free and due to applying the silicone-based skin contacting surface of a silicone-based material in accordance with the invention to the tattoo site as a pre-treatment to laser tattoo removal to remove or reduce, prior to laser tattoo removal, pre-existing scarring at the tattoo site created when the tattoo was applied.

The following examples illustrate various preferred embodiments of the invention, and these examples are not intended to be limiting.

### Example 1

A tattoo is applied to a person's skin. Then, immediately or soon after the tattoo is applied, a film of silicone-based gel is applied to the person's skin over and into contact with the tattoo at the tattoo site (that is, the site on the person's skin that the tattoo is located), preferably by dispensing the film of silicone-based gel from an Oleeva Scar Stick from Bio Med Sciences, Inc., containing a silicone-based gel having preferably greater than 50 w/w % polydimethylsiloxane onto the surface of the newly created tattoo to cover the tattoo. Application of the film of silicone-based gel over and into contact with the tattoo site is repeated preferably several times per day for at least 1 day and preferably for several days and more preferably for a week and even more preferably for two weeks and still more preferably for several weeks to enhance and improve the final outcome of the tattoo.

### Example 2

A tattoo is applied to a person's skin. Then, immediately or soon after the tattoo is applied, Oleeva Fabric, as described in my U.S. Pat. No. 7,087,135, from Bio Med Sciences, Inc. is applied to the surface of the newly created tattoo and maintained at the site for at least 1 day and preferably for several days and more preferably for about a week and even more preferably for two weeks and still even more preferably for several weeks to enhance and improve the final outcome of the tattoo.

### Example 3

A tattoo is applied to a person's skin. Then, immediately or soon after the tattoo is applied, Oleeva Clear, as described in my U.S. Pat. Nos. 5,656,279 and 5,980,923, from Bio Med Sciences, Inc. is applied to the surface of the newly created tattoo and maintained at the site for at least 1 day and preferably for several days and more preferably for a week and even more preferably for two weeks and still more preferably for several weeks to enhance and improve the final outcome of the tattoo.

### Example 4

A tattoo is applied to a person's skin. Then, immediately or soon after the tattoo is applied, a film of silicone-based gel is applied to the person's skin over and into contact with the surface of the newly created tattoo, preferably by dispensing the film of silicone-based gel from an Oleeva Scar Stick onto the surface of the newly created tattoo. Application of a film of silicone-based gel over and into contact with the newly created tattoo is repeated several times per day for at least 1 day and preferably for several days, at which time treatment of the tattoo site is continued by replacing the application of the film of silicone-based gel with the application of Oleeva Fabric, as described in my U.S. Pat. No. 7,087,135, or Oleeva Clear, as described in my U.S. Pat. Nos. 5,656,279 and 5,980,923, being applied to the surface of the tattoo site for at least 1 day and preferably for several days and more preferably for a week and even more preferably for two weeks and still more preferably for several weeks to enhance and improve the final outcome of the tattoo.

### Example 5

A tattoo is applied to a person's skin. Then, after the acute phase of healing (that is, after acute tattoo aftercare) at the tattoo site a film of silicone-based gel containing a silicone-based gel having preferably greater than 50 w/w % polydimethylsiloxane is applied onto the surface of the newly created tattoo to cover the tattoo during the prolonged phase of healing (that is, during prolonged aftercare) at the tattoo site. Application of the film of silicone-based gel over and into contact with the tattoo site is repeated preferably several times per day for at least 1 day and preferably for several days and more preferably for a week and even more preferably for two weeks and still more preferably for several weeks to enhance and improve the final outcome of the tattoo.

### Example 6

A tattoo is applied to a person's skin. Then, after the acute phase of healing (that is, after acute tattoo aftercare) at the tattoo site a self-adhesive silicone sheeting material (that is, a self-adhering silicone sheeting material) is applied to the surface of the tattoo and maintained at the site during the prolonged phase of healing (that is, during prolonged aftercare) at the tattoo site for at least 1 day and preferably for several days and more preferably for about a week and even more preferably for two weeks and still even more preferably for several weeks to enhance and improve the final outcome of the tattoo.

### Example 7

As a pre-treatment to laser tattoo removal, a film of silicone-based gel is applied over and into contact with the surface of the tattoo that is to be removed via laser tattoo removal, preferably by dispensing the film of silicone-based gel from an Oleeva Scar Stick from Bio Med Sciences, Inc. containing a silicone-based gel having preferably greater than 50 w/w % polydimethylsiloxane onto the surface of the tattoo to cover the tattoo. Application of the film of silicone-based gel over and into contact with the surface of the tattoo is repeated preferably several times per day for at least 1 day and preferably for several days and more preferably for about a week and even more preferably for two weeks and still even more preferably for several weeks prior to a laser tattoo removal procedure to enhance and improve the final outcome of the treatment site.

Then, after pre-treatment is completed and the silicone-based gel is removed from the tattoo, the tattoo is removed using laser tattoo removal, leaving the site from where the tattoo is removed in an optimal aesthetic condition due to being tattoo free and due to applying silicone-based tattoo dressings of the invention to the tattoo site as pre-treatment to laser tattoo removal to remove or reduce, prior to laser tattoo removal, pre-existing scarring at the tattoo site created when the tattoo was applied.

### Example 8

As a pre-treatment to laser tattoo removal, Oleeva Fabric, as described in my U.S. Pat. No. 7,087,135, from Bio Med Sciences, Inc. is applied to the surface of the tattoo that is to be removed via laser tattoo removal and maintained at the tattoo site for at least 1 day and preferably for several days and more preferably for about a week and even more preferably for two weeks and still even more preferably for several weeks prior to a laser tattoo removal procedure to enhance and improve the final outcome of the treatment site. Then, after pre-treatment is completed and the silicone-based tattoo dressing is removed from the tattoo, the tattoo is removed using laser tattoo removal, leaving the site from where the tattoo is removed in an optimal aesthetic condition due to being tattoo free and due to applying silicone-based tattoo dressings of the invention to the tattoo site as a pre-treatment to laser tattoo removal to remove or reduce, prior to laser tattoo removal, pre-existing scarring at the tattoo site created when the tattoo was applied.

### Example 9

As a pre-treatment to laser tattoo removal, Oleeva Clear, as described in my U.S. Pat. Nos. 5,656,279 and 5,980,923, from Bio Med Sciences, Inc. is applied to the surface of the tattoo that is to be removed via laser tattoo removal and maintained at the tattoo site for at least 1 day and preferably for several days and more preferably for about a week and even more preferably for two weeks and still even more preferably for several weeks, prior to a laser tattoo removal procedure, to enhance and improve the final outcome of the treatment site. Then, after pre-treatment is completed and the silicone-based tattoo dressing is removed from the tattoo, the tattoo is removed using laser tattoo removal, leaving the site from where the tattoo is removed in an optimal aesthetic condition due to being tattoo free and due to applying silicone-based tattoo dressings of the invention to the tattoo site as a pre-treatment to laser tattoo removal to remove or reduce, prior to laser tattoo removal, pre-existing scarring at the tattoo site created when the tattoo was applied.

### Example 10

As a pre-treatment to laser tattoo removal, a film of silicone-based gel is applied to the surface of the tattoo that is to be removed via laser tattoo removal, preferably by dispensing the film of silicone-based gel from an Oleeva Scar Stick onto the surface of the tattoo that is to be removed via laser tattoo removal. Application of the film of silicone-based gel over and into contact with the tattoo that is to be removed via laser tattoo removal is repeated preferably several times per day for at least 1 day and preferably for several days, at which time treatment of the tattoo site is continued by replacing the application of the film of silicone-based gel with the application of Oleeva Fabric, as described in my U.S. Pat. No. 7,087,135, or Oleeva Clear, as described in my U.S. Pat. Nos. 5,656,279 and 5,980,923, being applied to the surface of the tattoo site for at least 1 day and preferably for several days and more preferably for about a week and even more preferably for two weeks and still even more preferably for several weeks, prior to a laser tattoo removal procedure to enhance and improve the final outcome of the treatment site. Then, after pre-treatment is completed and the silicone-based tattoo dressing is removed from the tattoo, the tattoo is removed using laser tattoo removal, leaving the site from where the tattoo is removed in an optimal aesthetic condition due to being tattoo free and due to applying silicone-based tattoo dressings of the invention to the tattoo site as a pre-treatment to laser tattoo removal to remove or reduce, prior to laser tattoo removal, pre-existing scarring at the tattoo site created when the tattoo was applied.

### Example 11

As a pre-treatment to laser tattoo removal, a film of silicone-based gel is applied to the surface of the tattoo that is to be removed via laser tattoo removal, preferably by dispensing the film of silicone-based gel from an Oleeva Scar Stick onto the surface of the tattoo that is to be removed via laser tattoo removal. Application of the film of silicone-based gel over and into contact with the tattoo that is to be removed via laser tattoo removal is repeated preferably several times per day for at least 1 day and preferably for several days, at which time treatment of the tattoo site is continued by replacing the application of the film of silicone-based gel with the application of a self-adhesive silicone-based sheeting (that is, a self-adhering silicone sheeting) being applied to the surface of the tattoo site for at least 1 day and preferably for several days and more preferably for about a week and even more preferably for two weeks and still even more preferably for several weeks, prior to a laser tattoo removal procedure to enhance and improve the final outcome of the treatment site. Then, after pre-treatment is completed and the silicone-based tattoo dressing is removed from the tattoo, the tattoo is removed using laser tattoo removal, leaving the site from where the tattoo is removed in an optimal aesthetic condition due to being tattoo free and due to applying silicone-based tattoo dressings of the invention to the tattoo site as a pre-treatment to laser tattoo removal to remove or reduce, prior to laser tattoo removal, pre-existing scarring at the tattoo site created when the tattoo was applied.

In general, as exemplified in Examples 1-11 above and used throughout this application, a silicone-based material having a silicone-based skin contacting surface is maintained at the site of the tattoo (that is, is maintained at the tattoo site with the silicone-based skin contacting surface covering and being in contact with the tattoo) throughout the course of acute and prolonged tattoo aftercare, as well as throughout the course of pretreatment for tattoo removal via laser tattoo removal, including when needed, such as for hygienic reasons, removing any such silicone-based material already applied to and in contact with the tattoo and subsequently applying replacement silicone-based material having a silicone-based contacting surface to the tattoo site such that the silicone-based contacting surface of the replacement silicone-based material is placed over and into contact with the tattoo. The removal of silicone-based material from the tattoo and subsequent application of replacement silicone-based material onto the tattoo occurs as needed to maintain a hygienic environment at the tattoo site.

As exemplified in Examples 1-6 and throughout this application, my invention includes a composition comprising a silicon-based material (including silicone-based material in the form of a silicone-based film, or a silicone-based ointment, or a silicone-based liquid, or a silicone-based paste, or a silicone-based sheeting, or silicone, or polydimethylsiloxane, or polyorganosiloxane, or silicone gel, or silicone elastomer) for use in a method for the treatment of a tattoo to improve the outcome of the tattoo, wherein the composition is applied over and into direct contact with the tattoo immediately or soon after the tattoo is applied to a person's skin, and the composition is maintained in contact with the tattoo for a period of time of at least one day to several weeks, leaving the tattoo in optimal aesthetic condition having better (e.g., sharper and cleaner) colors in the tattoo than tattoos that are not treated in accordance with the invention, thereby enhancing and improving the final outcome of the tattoo.

As exemplified in Examples 7-11 and throughout this application, my invention includes a composition comprising a silicon-based material (including silicone-based material in the form of a silicone-based film, or a silicone-based ointment, or a silicone-based liquid, or a silicone-based paste, or a silicone-based sheeting, or silicone, or polydimethylsiloxane, or polyorganosiloxane, or silicone gel, or silicone elastomer) for use in a method for the treatment of an existing tattoo to remove the existing tattoo from a person's skin, wherein the composition is applied over and into contact with the existing tattoo, and the composition is maintained in contact with the existing tattoo for a period of at least one day to several weeks, and after maintaining the composition in contact with the existing tattoo for a period of at least one day to several weeks, the existing tattoo is removed using laser tattoo removal, leaving the site from where the tattoo has been removed in optimal aesthetic condition.

As used herein, the terms “self-adhesive” and “self-adhering” when used to describe silicone sheeting material and silicone sheeting mean that the silicone sheeting material and the silicone sheeting gently cling to the tattoo site and surrounding skin, but are easily removed when desired. The use of a separate adhesive is not necessary to hold such silicone sheeting material and such silicone sheeting in place at the tattoo site.

1. A method, comprising the steps of
applying a tattoo to a person's skin,
immediately or soon after the tattoo is applied, applying a silicone-based material over and into direct contact with the tattoo, and
maintaining a silicone-based material in contact with the tattoo for a period of time of at least one day.

2. The method of claim 1,
the period of time being several days.

3. The method of claim 1,
the period of time being a week.

4. The method of claim 1,
the period of time being two weeks.

5. The method of claim 1,
the period of time being several weeks.

6. The method of claim 1,
wherein maintaining the silicone-based material in contact with the tattoo includes removing any silicone-based material already applied to and in contact with the tattoo and then subsequently applying replacement silicone-based material over and into contact with the tattoo.

7. The method of claim 6,
wherein the removal of and subsequent application of silicone-based material occurs several times per day.

8. The method of claim 1,
wherein maintaining the silicone-based material in contact with the tattoo includes removing any silicone-based material already applied to and in contact with the tattoo and then subsequently applying a self-adhesive silicone-based sheeting over and into contact with the tattoo, the self-adhesive silicone-based sheeting having a silicone-based skin contacting surface that contacts the tattoo.

9. The method of claim 8,
wherein the self-adhesive silicone-based sheeting placed over and into contact with the tattoo is removed from the tattoo and subsequently replaced with replacement self-adhesive silicone-based sheeting several times per day.

10. The method of claim 1,
the silicone-based material being silicone, polydimethylsiloxane, polyorganosiloxane, silicone gel, silicone elastomer, or a silicone IPN.

11. A method of removing an existing tattoo from a person's skin, comprising the steps of
applying a silicone-based material over and into contact with the existing tattoo,
maintaining a silicone-based material in contact with the tattoo for a period of time of at least one day, and
after maintaining a silicone-based material in contact with the tattoo for a period of time, removing the tattoo using laser tattoo removal.

12. The method of claim 11,
the period of time being several days.

13. The method of claim 11,
the period of time being a week.

14. The method of claim 11,
the period of time being two weeks.

15. The method of claim 11,
the period of time being several weeks.

16. The method of claim 11,
wherein maintaining the silicone-based material in contact with the tattoo includes removing any silicone-based material already applied to and in contact with the tattoo and then subsequently applying replacement silicone-based material over and into contact with the tattoo.

17. The method of claim 16,
wherein the removal and subsequent application of silicone-based material occurs several times per day.

18. The method of claim 16, wherein the replacement silicone-based material subsequently applied over and into contact with the tattoo is a self-adhesive silicone-based sheeting.

19. The method of claim 18,
wherein the self-adhesive silicone-based sheeting placed over and into contact with the tattoo is removed from the tattoo and subsequently replaced with replacement self-adhesive silicone-based sheeting several times per day.

20. The method of claim 11,
the silicone-based material being silicone, polydimethylsiloxane, polyorganosiloxane, silicone gel, silicone elastomer, or a silicone IPN.

21. A method of treating a newly created tattoo, comprising the steps of
applying a silicone-based material over and into contact with the newly created tattoo, and
maintaining a silicone-based material in contact with the newly created tattoo for a period of time of at least one day.

22. The method of claim 21,
the period of time being several days.

23. The method of claim 21,
the period of time being a week.

24. The method of claim 21,
the period of time being two weeks.

25. The method of claim 21,
the period of time being several weeks.

26. The method of claim 21,
wherein the period of time begins upon completion of an acute phase of healing at the tattoo site, and wherein the period of time extends between 1 day and several weeks.

27. The method of claim 21,
wherein maintaining the silicone-based material in contact with the tattoo includes removing any silicone-based material already applied to and in contact with the tattoo and then subsequently applying replacement silicone-based material over and into contact with the tattoo.

28. The method of claim 27,
wherein the removal and subsequent application of silicone-based material occurs several times per day.

29. The method of claim 27,
wherein the replacement silicone-based material subsequently applied over and into contact with the tattoo is a self-adhesive silicone-based sheeting.

30. The method of claim 29,
wherein the self-adhesive silicone-based sheeting placed over and into contact with the tattoo is removed from the tattoo and subsequently replaced with replacement self-adhesive silicone-based sheeting several times per day.

31. The method of claim 21,
the silicone-based material being silicone, polydimethylsiloxane, polyorganosiloxane, silicone gel, silicone elastomer or, silicone IPN.

32. A method, comprising the steps of
applying a tattoo to a person's skin,
upon completion of an acute phase of healing at the tattoo site, applying a silicone-based material over and into direct contact with the tattoo during a prolonged phase of healing at the tattoo site, and
maintaining a silicone-based material in contact with the tattoo for a period of time of at least one day.

33. The method of claim 32,
the period of time being several days.

34. The method of claim 32,
the period of time being a week.

35. The method of claim 32,
the period of time being two weeks.

36. The method of claim 32,
the period of time being several weeks.

37. The method of claim 32,
wherein maintaining the silicone-based material in contact with the tattoo includes removing any silicone-based material already applied to and in contact with the tattoo and then subsequently applying replacement silicone-based material over and into contact with the tattoo.

38. The method of claim 37,
wherein the removal of and subsequent application of silicone-based material occurs several times per day.

39. The method of claim 32,
wherein maintaining the silicone-based material in contact with the tattoo includes removing any silicone-based material already applied to and in contact with the tattoo and then subsequently applying a self-adhesive silicone-based sheeting over and into contact with the tattoo, the self-adhesive silicone-based sheeting having a silicone-based skin contacting surface that contacts the tattoo.

40. The method of claim 39,
wherein the self-adhesive silicone-based sheeting placed over and into contact with the tattoo is removed from the tattoo and subsequently replaced with replacement self-adhesive silicone-based sheeting several times per day.

41. The method of claim 32,
the silicone-based material being silicone, polydimethylsiloxane, polyorganosiloxane, silicone gel, silicone elastomer, or a silicone IPN.
